(19) United States
(12) Patent Application Publication
(10) Pub. No.: US 2019/0167131 A1
(43) Pub. Date: Jun. 6, 2019

(54) HEART SOUND AND PULSE WAVEFORM ACQUISITION AND ANALYSIS

(71) Applicant: CALIFORNIA INSTITUTE OF TECHNOLOGY, Pasadena, CA (US)

(72) Inventors: Derek Rinderknecht, Arcadia, CA (US); Morteza Gharib, Altadena, CA (US); Niema Pahlevan, Pasadena, CA (US); Peyman Tavallali, Pasadena, CA (US)

(21) Appl. No.: 16/272,750

(22) Filed: Feb. 11, 2019

Related U.S. Application Data
(63) Continuation of application No. 15/199,160, filed on Jun. 30, 2016, now abandoned.
(60) Provisional application No. 62/187,777, filed on Jul. 1, 2015.
(60) Provisional application No. 62/187,772, filed on Jul. 1, 2015.

(51) Int. Cl. A61B 5/024; A61B 7/04; A61B 7/00

(52) U.S. Cl. CPC A61B 5/02438; A61B 5/02416; A61B 5/6831; A61B 7/00; A61B 2562/0223; A61B 7/04

(57) ABSTRACT

Devices, systems, and methods are described for acquiring heart sound timing content to enhance pulse waveform analysis.

### CROSS-REFERENCE TO RELATED APPLICATIONS

This filing is a continuation of U.S. patent application Ser. No. 15/199,160, filed Jun. 30, 2016, which claims the benefit of and priority to U.S. Provisional Application No. 62/187,772, filed Jul. 1, 2015 and U.S. Provisional Application No. 62/187,777, filed Jul. 1, 2015, all of which are incorporated by reference herein in their entireties for all purposes.

### FIELD

Devices, systems, and methods are described for acquiring heart sound timing content for pulse waveform and associated analysis.

### BACKGROUND

The field of noninvasive cardiovascular measurement techniques is growing. In this regard, there is a growing need for systems, devices, and methods capable of collecting a number of noninvasive physiological signals. These signals can be used to diagnose and even predict a number of physiological conditions, such as disease conditions and conditions associated with a healthy cardiovascular system. Sounds produced by the cardiac system can also prove useful. The signature produced by the sounds of the heart can be useful in the diagnosis of disease conditions.

In another context, heart sounds can be used to provide timing information related to left ventricular ejection time, spanning the contraction of the left ventricle and aortic valve opening, to the closure of the aortic valve forming the Dicrotic Notch (DN) of a pulse waveform. DN location (e.g., as understood in terms of valve closure events and relating to the timing of systole within a pulse waveform) can be critical to a number of pulse waveform diagnostic methods. One such method is the Intrinsic Frequency (IF) method described in U.S. Pat. No. 9,026,193 incorporated herein by reference in its entirety for all purposes. There exists needs for systems, devices, and methods for acquiring and/or using heart sound timing content to enhance pulse waveform analysis.

### SUMMARY

Example embodiments for obtaining heart sound timing content and pulse waveforms are described. These embodiments can include a sensor for pulse waveform acquisition and an additional pressure sensitive device (e.g., a second sensor) for detecting so-called heart sound. Pulse waveforms, which relate to pressure driven expansion or blood flow resulting from the contraction of the heart and subsequent systemic interactions, can be sensed and recorded a number of ways. In one example, pulse waveform sensing is performed optically with a Light Emitting Diode (LED) and photodetector system. Examples of such systems are described in US Publication No. 2015/0297105 and International Publication No. WO 2015/112512, both of which are incorporated by reference herein in their entireties for all purposes. Embodiments of the pressure-sensitive heart sound detection device may include, but are not limited to, a piezo accelerometer, an electromagnetic microphone, a piezoresistive pressure sensor, an optical microphone and/or a displacement sensor.

Notably, non-invasive peripheral measurements of pulse waveform signals often lack many of the features (e.g., the time period of cardiovascular ejection) that can reveal important clinical information. In elderly patients (often the subgroup with the highest need for continuous diagnostic monitoring), pulse waveform features are often attenuated making the identification of the DN tedious at best. Introducing the measurement of the heart sound addresses this problem as further described below.

Even members of a younger population may have attenuated waveform features relative to the requirements for IF analysis. Such waveforms may, however, still contain valuable clinical information. Combining a microphone to measure heart sounds enables the use of many pulse waveform analysis algorithms.

The example embodiments may operate by first measuring a pulse waveform and then measuring one or more heart sounds, or by first measuring one or more heart sounds and then measuring a pulse waveform. Likewise, simultaneous or synchronized measurement can be performed in the example embodiments. The pulse waveform and heart sound detection can be done with a single sensor or a number of sensors.

The pulse waveform can be measured from a multitude of locations including, but not limited to, the following: radial artery, brachial artery, femoral artery, carotid artery, chest wall or a photoplethysmograph (PPG) location such as in connection with a finger, leg or elsewhere.

Using the start of the pulse wave and assuming the heart rate has not changed substantially, the heart sound can also be measured simultaneously or in close time proximity to the pulse waveform measurement at any of following locations: radial artery, brachial artery, femoral artery, carotid artery or chest wall. From the heart sound recording, the start of the cardiac cycle from the pulse waveform and the first heart sound (e.g., S1, that results from mitral and tricuspid valve closure occurring at the beginning of ventricular systole) can be used to align the waveforms, and DN location (e.g., notch time) can be estimated. In other cases where heart rates vary, the waveforms may be normalized with respect to time and/or amplitude such that all waveforms span similar ranges regardless of morphology.

An analog/digital stethoscope may be used to record the heart sounds on the chest or on the carotid artery. This timing information can be used to infer timing on a brachial pulse waveform, radial pulse waveform, or PPG waveform. The combinations of locations where this technique can be applied are not limited to those described above. A table below details a more extensive, but still non exhaustive list.

In some cases, the heart sounds may be recorded in the same location, area or vicinity of the pulse waveform. Likewise, the heart sounds may be recorded simultaneously with the pulse waveform. In one example, a wrist worn device is used where the pulse waveform is recorded from a PPG and a small microphone located on or within the band or watch itself is used to record the heart sound for determining timing information for the waveform.

The sensors may be integrated into a watch body or into a band (of a watch or otherwise). One wrist-band embodiment includes a multitude of sensors which may allow freedom of movement. In another wrist-band embodiment, a microphone and a pulse waveform sensor are located over the radial and ulnar arteries (or vice versa) with positioning maintained securely by the band. Either way, sounds and pulse waveform are recorded simultaneously and may be broadcast wirelessly.

The detection (of heart sound and/or pulse waveform) may be purely optical interaction with the skin or may be combined with a membrane (e.g., as further described in US Publication No. 2015/0297105 and International Publication No. WO 2015/112512 referenced above, and also described in U.S. Pat. No. 5,363,855, which is incorporated herein by reference in its entirety for all purposes). The membrane can serve to maintain similar photodetector response in a number of situations of changing light or optical spectrum.

Such a membrane may also possess certain features such as nodes, bumps, or curvature to assist in the collection of data. In one such embodiment, the membrane is formed to match the curvature of the wrist as well as provide more intimate contact between the membrane and the skin overlying the artery.

Of the band-based platform examples, one example is a wrist worn watch that combines pulse waveform detection and heart sound detection into a wearable platform. As for the “watch” description, this terminology may indicate function and/or form factor. Namely, the subject systems or devices may offer the full function of a watch, even a so-called “smartwatch” (e.g., as provided by Apple, Samsung, or others) together with the subject features in a wrist-worn package. Alternatively, the subject features are provided without a time-telling display and/or other associated features.

In other examples, the band-based device may instead be worn on the neck to measure information from the carotid artery. Another example is configured to substantially encircle a portion of a subject's appendage (e.g., the upper arm, waist, or thigh, etc.), or to encircle the entire appendage. As used herein, the term “encircle” and its variants do not require the structure to form a geometric circle around the body portion and, in fact, any shape can be used to extend around the body portion. Likewise, the term “surround” does not require the body portion to be surrounded in all directions, as such would be inconsistent with the usage of that term herein. Instead the terms “encircle” and “surround” are used in the same sense that a watch or a wrist-worn activity monitor encircles and surrounds the arm or wrist. Still other options are within the scope of this disclosure.

To encircle or surround the body portion, the band may be provided to the wearer (e.g., as provided in packaging) in an elongate shape or form and be closed to define the surrounding shape, e.g., a circle, oval, etc. This may be accomplished with one or more snaps, buckles, VELCRO elements, magnetic clasps or other mechanisms known in the art. Alternatively, the band may be manufactured and provided to the wearer in the surrounding curved shape (with or without a break) and may include diameter adjustment or adjustable features (e.g., elastic, air bladder or as in a spring and linkage or other type of expansion watch band).

An adhesive-type patch (e.g., similar to an ECG or EKG electrode patch) offers another form factor suitable for measuring heart sound and the pulse waveform. In connection with such a device (or otherwise), the pulse waveform and/or heart sound detection may also be done electromagnetically or via magnetoresistive materials.

Any of these devices and/or systems may have Bluetooth connection capability or otherwise be wireless. In certain situations, the hardware and/or associated methodology may be used for clinical monitoring in health care establishments (e.g., hospital departments like an operating room (OR), or intensive care unit (ICU)). In other cases, such sensors may be useful for ambulatory monitoring.

The subject devices or systems, kits in which they are included (with and without assembly), methods of use and manufacture (including assembly of the constituent components) are all within the scope of the present disclosure. Some aspects of the same are described above and more detailed discussion is presented in connection with the figures below.

Various systems, devices, methods, features and advantages of the subject matter described herein will be or will become apparent to one with skill in the art upon examination of the following figures and Detailed Description. It is intended that all such systems, devices, methods, features and advantages be included within this description, be within the scope of the subject matter described herein and be protected or protectable by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is a chart showing an example of each of a pulse waveform and heart sound from a young (e.g., up to about 30 years in age) human subject.

FIG. 1B is a chart showing an example of each of a pulse waveform and heart sound for an old (e.g., over about 65 years in age) subject.

FIG. 2 is a diagram presenting example locations for acquiring pulse waveform and heart sounds and providing a superimposed system output.

FIGS. 3A and 3B depict an example of an attenuated pulse waveform signal, and an example of a heart sound signal, respectively, each taken over more than one cycle in time with a large Y scale.

FIG. 4 is a diagram of an example embodiment of a system in which heart sound and a pulse waveform are recorded in separate locations.

FIG. 5 is a diagram of another example embodiment of a system in which heart sound and a pulse waveform are recorded in separate locations in connection with a first band-based embodiment.

FIG. 6 is a diagram of another example embodiment of a band-based system, together with combined heart sound and pulse waveform information recorded therewith.

FIG. 7 is an illustration of an example embodiment of a band-based system including additional sensors.

FIG. 8A is an illustration of an example embodiment of a band-based system with a single sensor location. FIG. 8B is a cross-sectional view of the embodiment in FIG. 8A taken along line 8B-8B.

FIG. 9 is an illustration of an example embodiment of a finger-worn band-based system.

FIG. 10 is an illustration of an example embodiment of a leg-worn band-based system.

FIG. 11 is an illustration of an example embodiment of a neck-worn band-based system.

FIG. 12 is an illustration of an example embodiment of a sensor patch based system.

FIGS. 13A and 13B are cross-sectional views of the embodiment of FIG. 12, taken along line 13-13, showing alternative sensor architecture embodiments.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

It should be noted that all features, elements, components, functions, acts and steps described with respect to any embodiment provided herein are intended to be freely combinable and substitutable with those from any other embodiment. If a certain feature, element, component, function, act or step is described with respect to only one embodiment, then it should be understood that that feature, element, component, function, act or step can be used with every other embodiment described herein unless explicitly stated otherwise. This paragraph therefore serves as antecedent basis and written support for the introduction of claims, at any time, that combine features, elements, components, functions, acts and steps from different embodiments, or that substitute features, elements, components, functions, acts and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

Various example embodiments are described below. Reference is made to these examples in a non-limiting sense. They are provided to illustrate more broadly applicable aspects of inventive aspects. Various changes may be made to the embodiments described and equivalents may be substituted without departing from their true spirit and scope. In addition, many modifications may be made to adapt a particular situation, material, composition of matter, process, process act(s) or step(s) to the objective(s), spirit or scope of the claims made herein.

As referenced above, the example embodiments described herein can be used to measure heart sound and a pulse waveform at various measurement locations. A non-exhaustive list of examples (indicated by an X) are set forth below in TABLE 1.

TABLE 1

| | | Location of Waveform Recording | | | | | | | | | | |
|---|---|---|---|---|---|---|---|---|---|---|---|---|
| | | | | | | | Dorsalis | | | | | |
| | | Carotid | Brachial | Radial | Femoral | | pedis | | Posterior | Finger | | |
| | | (Right | (Right | (Right | (Right | Popliteal | (Right | Temporal | Tibial | (Right | | Invasive |
| | | or | or | or | or | (Right or | or | (Right or | (Right or | or | Invasive | Hemodynamic |
| | | Left) | Left) | Left) | Left) | Left) | Left) | Left) | Left) | Left) | Catheter | Sensor |
| Location | Carotid (Right or | | x | x | x | x | x | x | x | x | x | x |
| of | Left) | | | | | | | | | | | |
| Heart | Brachial (Right or | x | | x | x | x | x | x | x | x | x | x |
| Sound | Left) | | | | | | | | | | | |
| Recording | Radial (Right or | x | x | | x | x | x | x | x | x | x | x |
| | Left) | | | | | | | | | | | |
| | Femoral (Right or | x | x | x | | x | x | x | x | x | x | x |
| | Left) | | | | | | | | | | | |
| | Popliteal (Right | x | x | x | x | | x | x | x | x | x | x |
| | or Left) | | | | | | | | | | | |
| | Dorsalis pedis | x | x | x | x | x | | x | x | x | x | x |
| | (Right or Left) | | | | | | | | | | | |
| | Temporal (Right | x | x | x | x | x | x | | x | x | x | x |
| | or Left) | | | | | | | | | | | |
| | Posterior Tibial | x | x | x | x | x | x | x | | x | x | x |
| | (Right or Left) | | | | | | | | | | | |
| | Finger (Right or | x | x | x | x | x | x | x | x | | x | x |
| | Left) | | | | | | | | | | | |
| | Invasive Catheter | x | x | x | x | x | x | x | x | x | | x |
| | Invasive | x | x | x | x | x | x | x | x | x | x | |
| | Hemodynamic | | | | | | | | | | | |
| | Sensor | | | | | | | | | | | |

The example embodiments described herein can be adapted for use with any of these locations (and others) and, to the extent a particular combination of hardware and location is not explicitly described herein, then the implementation of that embodiment can be accomplished by one of ordinary skill in the art after reading the present application. It is noted that some of these embodiments can be implemented with conventional “off-the-shelf” hardware (e.g., an invasive catheter or a hemodynamic sensor).

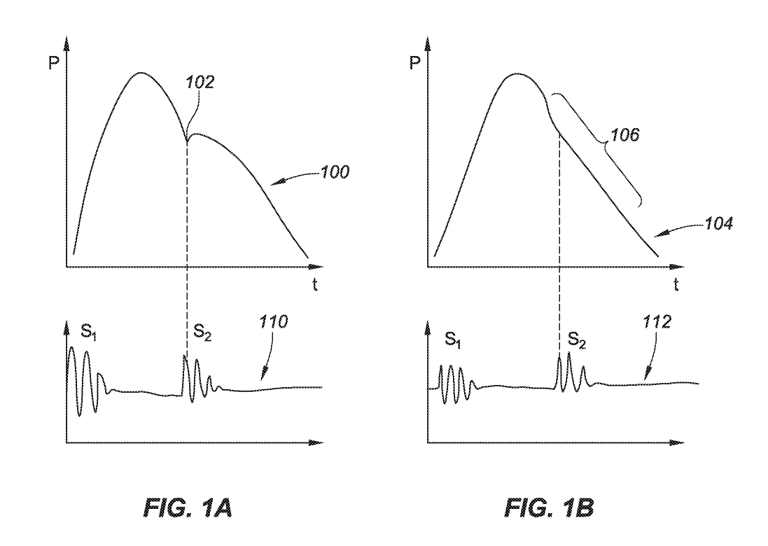

FIGS. 1A and 1B are illustrative with regard to the signals acquired and employed in a superposition method. In FIG. 1A, an example of a pulse waveform 100 is shown. It includes a well-defined or discernable DN.

FIG. 1A also shows a recorded heart sound signal 110 with first and second heart sounds (S1 and S2). Here, S1 aligns with the beginning of the pulse waveform. The beginning location of S2 along time axis t (as indicated by the dashed line) aligns with the DN.

FIG. 1B shows an example of an attenuated pulse waveform 104. A zone or region of uncertainty 106 exists over which the DN should be located. Using a recorded heart sound signal 112, an estimate of DN location can be determined at the time position indicated by the dashed line. With DN timing known relative to waveform 104, even an attenuated signal can be analyzed with IF and/or other analytical techniques. Such an analysis would not be possible with the pulse waveform alone (e.g., without the timing information provided by the heart sounds signal).

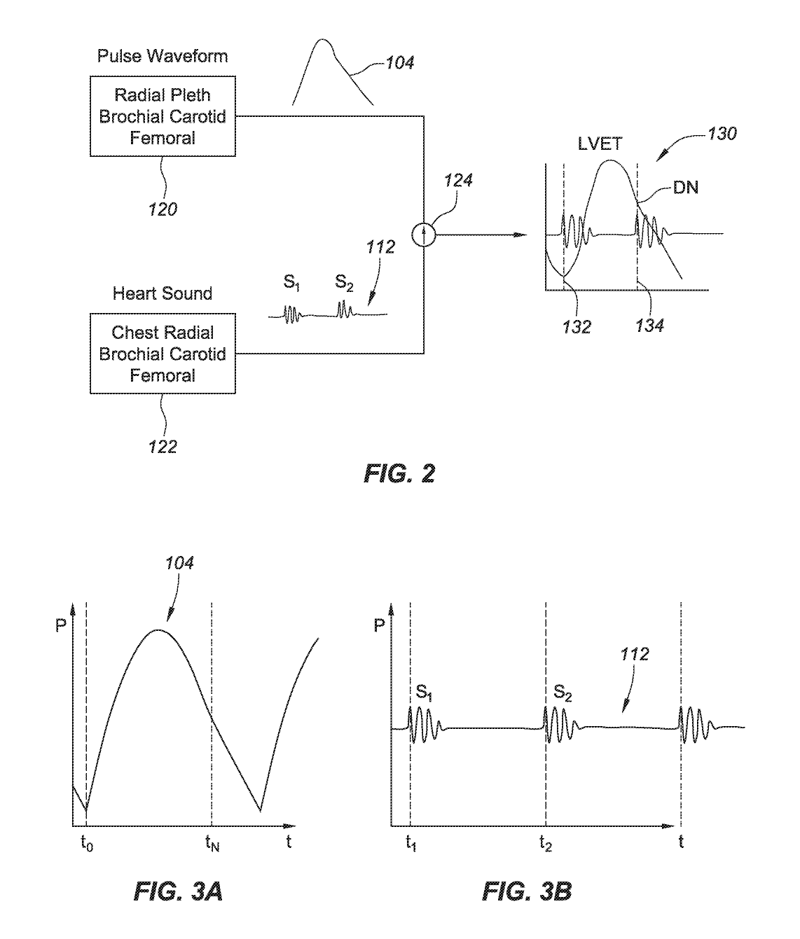

FIG. 2 is further illustrative of the method. Functional block 120 represents hardware to acquire (e.g., sense and record) a pulse pressure waveform (an attenuated example 104 in this case) over one or more cycles at any of the locations of TABLE 1 (or others). Functional block 122 represents use of (the same or different) hardware to acquire heart sounds S1 and S2, again, at any of a selection of the example locations listed.

Using computer processing circuitry, optionally in real time, the signals are superimposed by a process 124 to yield a result that may be electronically stored and subsequently analyzed, displayed (e.g., as in composite graph 130) or otherwise handled. In this graph, S1 and pressure waveform timing start is coincident along line 132. DN timing (as determined in the attenuated signal 112) along line 134.

FIGS. 3A and 3B depict an attenuated pulse waveform signal 104, and a heart sound signal 112, respectively, each taken over more than one cycle in time with a large Y axis scale. FIGS. 3A and 3B are helpful in illustrating example embodiments of methods of using the start of the cardiac cycle from the pulse waveform and S1 from the heart sound recording to align the waveforms to estimate Dicrotic Notch (DN) time. In these embodiments, if the heart rate when the pulse waveform is recorded (HR_{PW}) is equal to or about equal to the heart rate when the heart sounds are recorded (HR_{S}) or these signals are recorded simultaneously then t₀=t₁ or t₀≠t₁ then tₙ=t₀+(t₂−t₁) as shown. By this calculation (optionally referred to as superposition of the waveforms), tₙ provides an estimate of DN timing location. With this position, waveform 110 can then be analyzed using IF methodology. In other words, the start of the cardiac cycle from the pulse waveform and S1 from the heart sound can be used to align the waveforms so that DN time can be estimated.

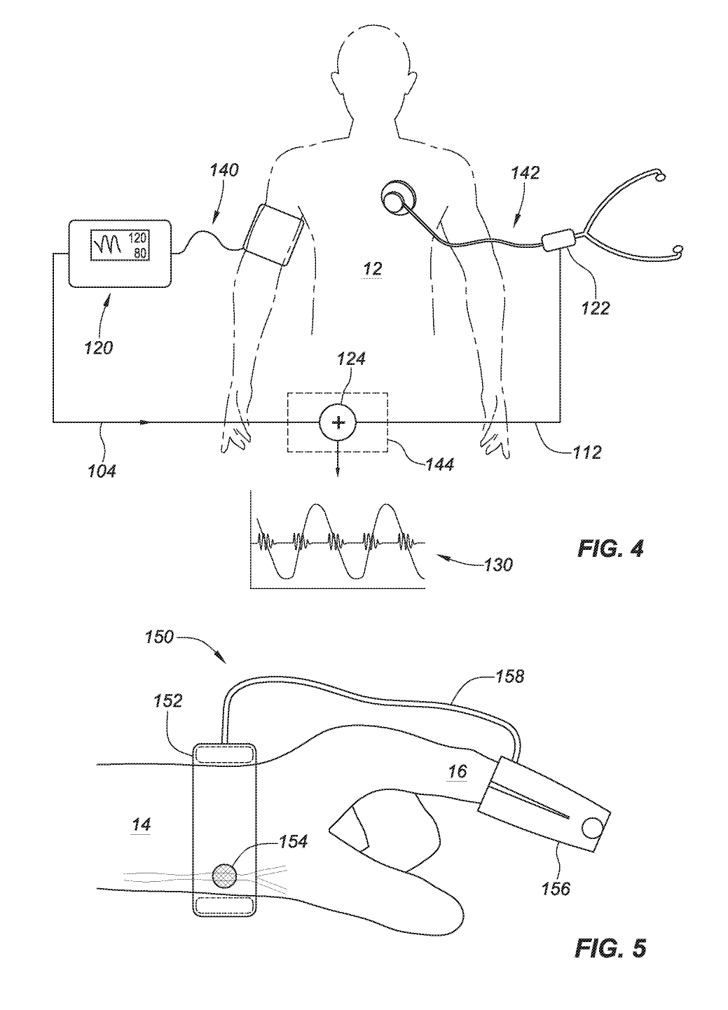

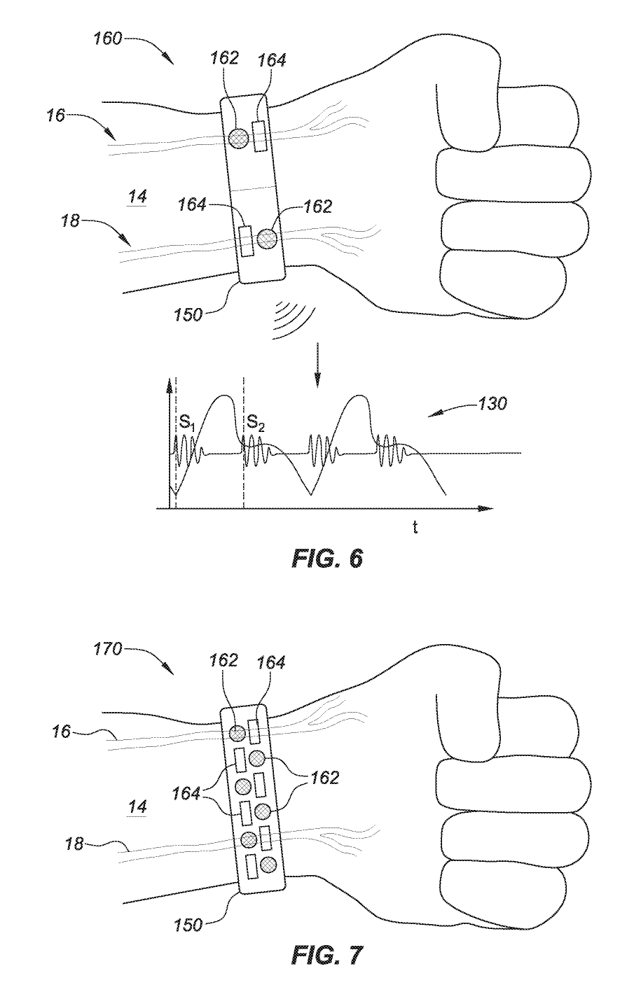

As shown in FIG. 4, functional modules 120 and 124 may be incorporated in existing hardware such as a digital blood pressure cuff 140 and a digital stethoscope 142. A separate hardware piece (such as a smart phone, a general purpose computer or other hardware) 144 may incorporate function block 124. In any case, signals corresponding to a pulse waveform 104 (here, the brachial waveform of a subject 10) and heart sounds 112 (here, recorded at the chest wall 12 of the subject) are received and combined or otherwise superimposed to provide composite timing and DN information, optionally, as shown as graph 130. Otherwise, the signal data and systolic and diastolic intervals may be simply stored electronically in a spreadsheet or other means for subsequent IF calculation purposes.

In any case, hardware pictured in FIG. 4 and associated methodology contemplates acquiring and recording pulse waveform and heart sound signals at different locations with different sensors and combining or otherwise superimposing their information.

Another approach to utilizing this approach is shown in FIG. 5. Here, system 150 includes a band 152 located around the wrist 14 of a user incorporates or carries a sensor 154 positioned to measure heart sounds at the subject's radial artery 16. A PPG or pulse oximeter 156 is set over the subject's finger 16 to measure the pulse waveform. The PPG 156 is optionally connected by an electrical lead 1568 to band 150 to operate much like the system shown in in FIG. 4. In this regard, the band may further incorporate electronic hardware 144 to perform function 124.

FIG. 5 shows another band-based embodiment 160, which is wireless (see signal icon). On-board electronics may communicate a composite graph or combined signal 130 as indicated by the arrow in the figure. Alternatively, pulse and heart sound signals may be communicated wirelessly to external hardware for superposition and/or other (e.g., IF calculation) processing.

Regardless, embodiment 160 includes a band 150 in which at least one pair of sensors are included. The paired sensor may include a microphone 162 for sensing heart sound and an optical sensor 164 for detecting the pulse waveform. One such pair of sensors may be located so that it picks-up a signal from a subject's radial artery 14, with another pair of the sensors located to pick-up a signal from the ulnar artery 18. Alternatively, the sensor pair (162 and 164) may be broken-up so that one sensor (162 or 164) is positioned (by the band) over the radial artery and the other sensor (164 or 162) over the ulnar artery. When two pairs of sensors are used as illustrated, signal acquisition redundancy is provided. The sensors may be multiplexed or sampled sequentially. Different orientations or array grids may be used as shown in 162 and 164 to optimize signal fidelity and signal-to-noise ratio.

In the band-based embodiment 170 shown in FIG. 7, the sensor pairs are multiplexed, with the number of sensors multiplied to make sensing further redundant. As such, the band is allowed freedom of motion while still ensuring good signal acquisition.

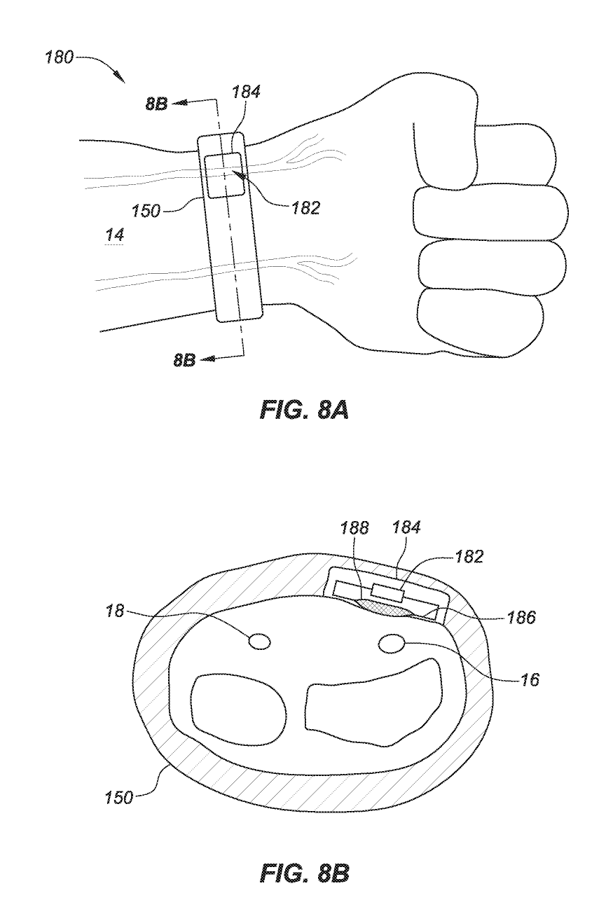

In contrast, a single sensor embodiment 180 in shown in FIGS. 8A and 8B. Here, the sensor 182 set within a cell or pocket 184 of the band may be purely optical, having interaction with the skin or it may operate with a membrane 186. As shown, the membrane includes a raised region, protuberance or bump 186 to match the curvature of the wrist (helping hold or “lock” the band in position) as well as to provide more intimate contact between the membrane and the skin overlying the artery. When the membrane is a plastic film, the geometry of the bump may be provided by thermoforming. Other options are possible as well such as molding using soft materials, machining or techniques such as three dimensional (3D) printing.

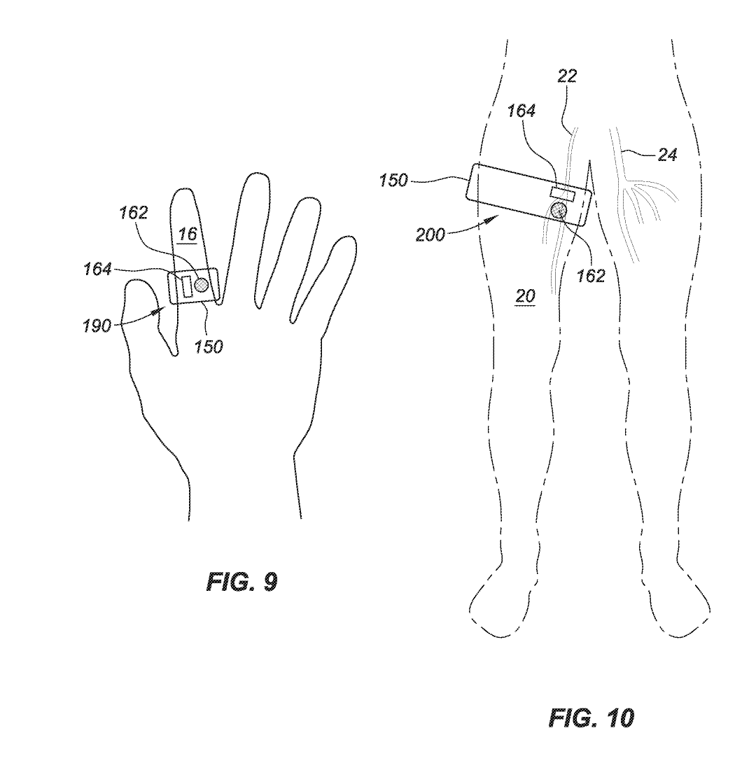

FIG. 9 shows a band-based embodiment 190 in the form of a ring, set upon a user's finger 16. FIG. 10 shows a band-based embodiment 200 over a subject's leg 20 to sense at or over the left femoral artery 22. This band 150 is configured and/or positioned like a bridal garter. Alternatively (or additionally) the right femoral artery 24 can have device 200 set there for sensing.

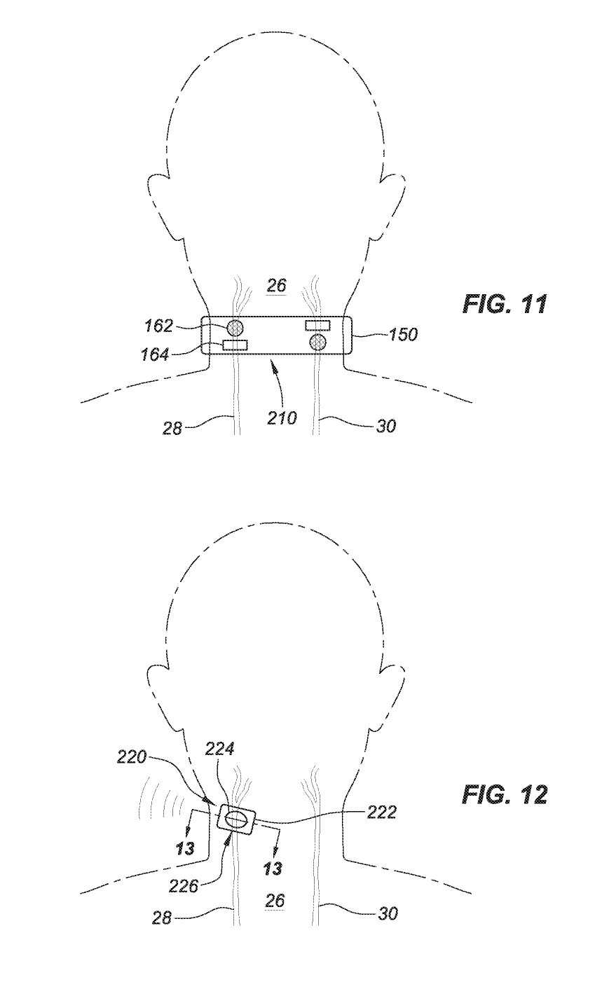

FIG. 11 shows a band-based device 210 configured to be worn on the neck 26 to measure on the right and or left carotid arteries (28 and 30, respectively). Here, (again located on the neck 26 of a subject) a sensor device 220 may take the form of an adhesive patch as illustrated in FIG. 12. The patch includes a housing 222 having a sensor region 224 and an underlying adhesive region 226. The adhesive may be in the form of an applied patch or be otherwise provided. It may be protected by a peel ply (not shown) prior to its preparation for use. The membrane and/or the adhesive portion of the patch may also be disposable allowing the user to retain the sensor, signal conditioning hardware, and data transmission hardware, but replace elements in contact with the skin to maintain sterility and cleanliness.

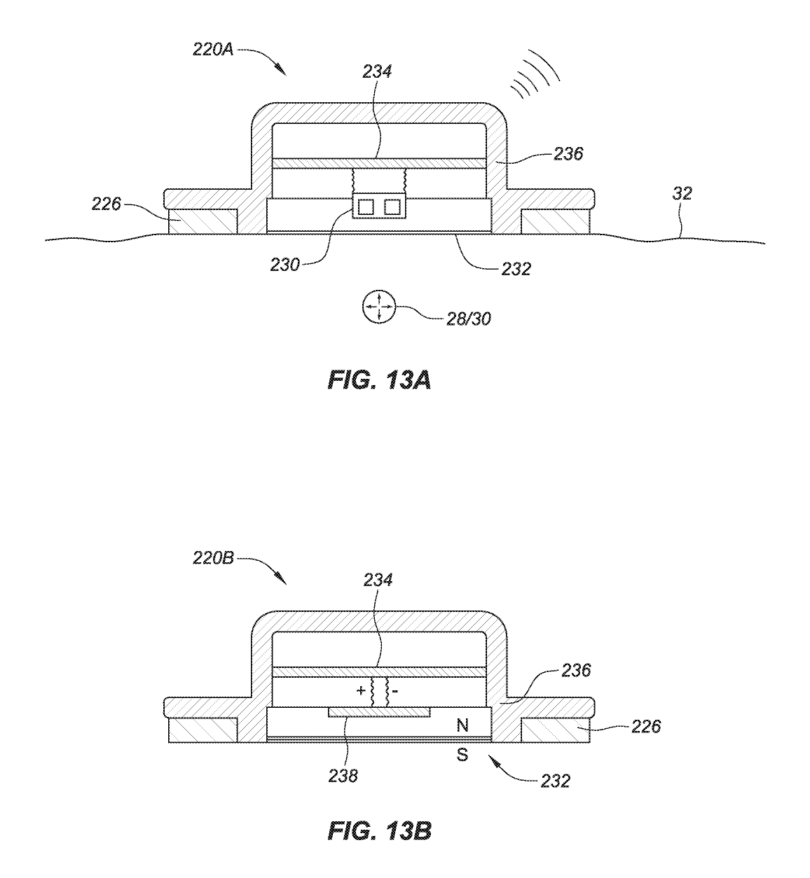

In version 220A shown in FIG. 13A, an optical sensor 230 is provided to work in association with membrane 232. Electronics 234 may be included in space of body 236 supporting or above the region of sensor 230. As shown, the adhesive element 224 may be in the form or a ring surrounding the membrane 232 (where this “ring” may be round, rectangular or otherwise shaped). Alternatively, the membrane may be lightly coated with adhesive itself.

In any case, with the patch temporarily adhered to the skin 32 of a subject, the pulse waveform and sounds carried within an artery (e.g., carotid 28 or 30) can be acquired by a single sensor. Alternatively, different sensors may be provided as per above. The signals may be processed on-board by the electronics and communicated wirelessly (as indicated), or simply recorded and communicated wirelessly as described above.

FIG. 13B shows an embodiment version 220B in which the membrane 232 is magnetically poled (see the North (N) and South (S) pole indication (which, of course, may be reversed) and a magnetoresistive or hall effect sensor 238, the material of which changes voltage in response to the displacement of the membrane. Notably, the patch-based embodiment 220 (irrespective of its inner workings per variation 220A or 220B) may be placed on the neck 26 as described above, on or at the wrist 14 or otherwise.

Additional Variations

The band may comprise any conventional material including metals, rubbers, plastics, and/or so-called “smart” materials that can be controlled via external stimuli. Likewise, other system components may be constructed with commonly available components and/or materials as will be appreciated by those with skill in the art.

In addition to the embodiments disclosed already, still more variations are within the scope of this description. For example, the various illustrative methods or processes described in connection with the embodiments herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein.

A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a digital camera of any type including those using CMOS, CCD or other digital image capture technology.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available non-transitory media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

To the extent the embodiments disclosed herein include or operate in association with memory, storage, and/or computer readable media, then that memory, storage, and/or computer readable media are intended to be non-transitory. Accordingly, to the extent that memory, storage, and/or computer readable media are covered by one or more claims, then that memory, storage, and/or computer readable media is only non-transitory.

Operations as described herein can be carried out on or over a website or network. The website can be operated on a server computer or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Moreover, no limitations from the specification are intended to be read into any claims, unless those limitations are expressly included in the claims. The computers described herein may be any kind of computer, either general purpose, or some specific purpose computer such as a workstation. The programs may be written in C, or Java, Brew or any other programming language. The programs may be resident on a storage medium, e.g., such as those already described. The programs may also be run over a network, for example, with a server or other machine sending signals to the local machine, which allows the local machine to carry out the operations described herein.

As used herein and in the appended claims, the singular forms “a”, “an”, and “the” include plural referents unless the context clearly dictates otherwise. In other words, use of the articles allow for “at least one” of the subject items in the description above as well as the claims below. The claims may exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The subject matter described herein and in the accompanying figures is done so with sufficient detail and clarity to permit the inclusion of claims, at any time, in means-plus-function format pursuant to 35 U.S.C. section 112, part (f). However, a claim is to be interpreted as invoking this means-plus-function format only if the phrase “means for” is explicitly recited in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. A band-based apparatus for heart sound and pulse waveform acquisition, the apparatus comprising:
an elongate band adapted to encircle a human body part;
a first sensor positioned along the band and adapted to measure heart sound; and
a second sensor different from the first sensor, wherein the second sensor is adapted to measure a pulse waveform.

2. The apparatus of claim 1, wherein the band is a wrist band and the second sensor is configured as a finger sensor and connected to the band by electrical leads.

3. The apparatus of claim 1, wherein the second sensor is also positioned along the band.

4. The apparatus of claim 3, further comprising at least one additional first sensor and at least one additional second sensor.

5. The apparatus of claim 4, further comprising an inward-facing bump adapted to stabilize position of the band.

6. The apparatus of claim 1, wherein the band is a finger band or a neck band.

7. The apparatus of claim 1, further comprising:
processing circuitry communicatively coupled with the first and second sensors; and
a non-transitory memory on which is stored a plurality of instructions that, when executed, cause the processing circuitry to determine a Dicrotic Notch timing position for the pulse waveform provided by the second sensor by superposition of the heart sound provided by the first sensor and the pulse waveform.

8. A patch-based apparatus for heart sound and pulse waveform acquisition, the apparatus comprising:
a body;
an adhesive region adapted to attach the body to a subject's skin; and
at least one sensor adapted to acquire heart sound and a pulse waveform.

9. The apparatus of claim 8, wherein the sensor is an optical sensor.

10. The apparatus of claim 8, wherein the sensor is a magnetoresitive sensor.

11. The apparatus of claim 8, further comprising:
processing circuitry communicatively coupled with the at least one sensor; and
a non-transitory memory on which is stored a plurality of instructions that, when executed, cause the processing circuitry to determine a Dicrotic Notch timing position for the pulse waveform provided by the at least one sensor by superposition of the heart sound provided by the at least one sensor and the pulse waveform.

12. A method for heart sound and pulse waveform acquisition and analysis, the method comprising:
acquiring a pulse waveform at a first location;
acquiring heart sound at a second location; and
determining a Dicrotic Notch timing position for the pulse waveform by superposition of the pulse waveform and the heart sound.

13. The method of claim 12, wherein the first location is selected from a radial artery, brachial artery, a femoral artery, a carotid artery, chest wall and a photoplethysmograph location.

14. The method of claim 12, wherein the second location is selected from a radial artery, a brachial artery, a femoral artery, a carotid artery and chest wall.

15. The method of claim 14, wherein the first location is different from the second location.

16. The method of claim 12, further comprising:
acquiring a pulse waveform at a first location with at least one sensor;
acquiring heart sound at a second location with the at least one sensor; and
determining, with processing circuitry, a Dicrotic Notch timing position for the pulse waveform by superposition of the pulse waveform and the heart sound.

17. The method of claim 16, wherein the a first sensor is used to acquire the pulse waveform and a different, second sensor is used to acquire the heart sound.

18. The method of claim 12, performed with the apparatus described in claim 2.

19. The method of claim 12, performed with the apparatus described in claim 3.

20. The method of claim 12, performed with the apparatus described in claim 4.

21-23. (canceled)
